(19) United States
(12) Patent Application Publication
(10) Pub. No.: US 2008/0157550 A1
(43) Pub. Date: Jul. 3, 2008

(54) MULTI-TOOL TWEEZER

(76) Inventors: Patrick Burgess, Jalisco (MX); Tylor Garland, Studio City, CA (US); Aldis Rauda, West Hollywood, CA (US); Stan Chudzik, Alpharetta, GA (US); Christin Hartsfield, Atlanta, GA (US)

Correspondence Address: GARDNER GROFF GREENWALD & VILLANUEVA, PC, 2018 POWERS FERRY ROAD, SUITE 800, ATLANTA, GA, 30339, US

(21) Appl. No.: 11/928,439

(22) Filed: Oct. 30, 2007

Related U.S. Application Data
(63) Continuation-in-part of application No. 29/275,604, filed on Dec. 30, 2006, now Pat. No. D559459.

(51) Int. Cl. B25B 7/00; A61B 17/50

(52) U.S. Cl. 294/99.2; 606/211

(57) ABSTRACT

The present disclosure is generally directed to a multi-tool with more than one tweezer.

### RELATED APPLICATION

This continuation-in-part application claims the priority benefit of U.S. Design Patent application Ser. No. 29/275,604, filed on Dec. 30, 2006, the contents of which are incorporated by reference as if fully expressed fully herein.

### FIELD OF THE DISCLOSURE

The present disclosure is generally directed to a multi-tool with more than one tweezer.

### BACKGROUND OF THE DISCLOSURE

Most types of tweezers that are generally of a uniform size and are designed for use by women. These types of tweezers may not be suitable for use by men. Because current tweezers are designed mainly with women's ergonomic/human factors measurements in mind, meaning that they are designed for smaller hands and fingers. The small size of tweezers may make it more difficult for use by men because they typically have larger fingers and hands. Moreover, the amount of pressure exerted to operate cosmetic tweezers is also designed for use by women.

Additionally, most commercially available tweezers are designed for grooming rather than the removal of splinters or other debris that may lodge in the skin. Men are more likely to use tweezers to remove debris that is lodged in the skin that are the result of manual labor. The present invention was conceived to alleviate this problem by providing a multi-tool that is specifically designed for use by men in the situations that are most applicable to men.

### SUMMARY OF THE INVENTION

The present invention is directed to a multi-tool with more than one side where the multi-tool is designed to dig, pull, squeeze, magnify and/or illuminate. In one embodiment of the present invention the multi-tool may include two plates joined by a spring. In this embodiment, the spring force between the two plates may be about 1.0 to about 2.5 lbs. In a related embodiment, the dig functionality may be performed by a tweezer tip with a sharp end. In still another embodiment of the present invention, the pull functionality may be performed by a flat perpendicular pulling tweezer. In another embodiment, the squeeze functionality may be performed by a rounded end tweezer. In a related embodiment, the illuminate function may be performed by a light. Additionally, the light used in the illuminate function may be adapted to pivot.

In an embodiment of the present invention, the two plated may have a surface coating for better grip.

### BRIEF DESCRIPTION OF THE DRAWINGS

FIG. 1 is a top view of one embodiment of the present invention.

FIG. 2 is a side view of the embodiment shown in FIG. 1.

While the method and device described herein are susceptible to various modifications and alternative constructions, certain illustrative embodiments thereof have been shown in the drawings and will be described below in detail. It should be understood, however, that there is no intention to limit the invention to the specific forms disclosed, but on the contrary, the intention is to cover all modifications, alternative constructions, and equivalents falling within the spirit and scope of the disclosure.

### DETAILED DESCRIPTION OF THE DISCLOSURE

The present invention solves the problems listed above by combining multiple specific functions into one tweezer-like apparatus. Any number of functions may be combined and different functions may be used in various embodiments and variations

In a specific embodiment of the present invention, three functions may be combined and may be defined by their respective uses. The functions may include magnification, illumination, pulling, digging or squeezing. Pulling, digging, and squeezing may be performed my tweezers of different size and/or angle. In some embodiments, a function may be repeated using different size tweezers. The tool preferably has multiple sides and may be in the shape of a triangle, square, pentagon, hexagon or any other multi-sided geometric. The present invention seeks to combine multiple functions in a handy multi-tool that is preferably pocket-sized.

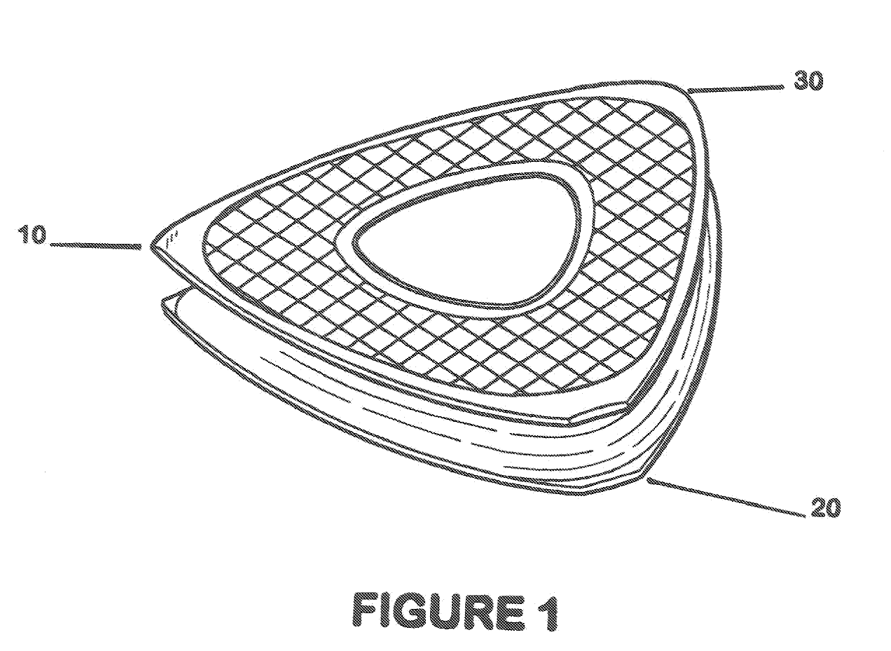

In one embodiment, a desired function or use may be to remove splinters by a digging motion. Referring to FIG. 1, tip 10 represents a tip that may be used to remove splinters. In this embodiment, the tweezer tip may be very sharp and may allow the user to dig into the skin to remove wood, metal, glass, or other debris imbedded into the skin. This same tip may also be used to pluck fine hairs.

In another embodiment of the present invention, one function or use may be to pull, for example, to remove debris or hair from the skin. This tip is preferably designed as a flat perpendicular pulling tweezer, as depicted in FIG. 1 as tip 20. Tip 20 allows the user to remove debris from the skin or pluck hair in areas such as the eyebrow, nose, and ear.

In another embodiment, a function, shown as tip 30, may be to squeeze. Tip 30 may be identified by a rounded shape that is useful in squeezing or pinching the skin. Tip 30 may allow the user to squeeze skin afflictions such as embedded debris or minor blisters to remove the fluid trapped between the skin layers. Tip 30 may also allow one to pinch minor skin blood clots for the removal of trapped blood or puss due to injury or to remove blemishes.

In another embodiment, the multi-tool may possess two different sizes of a specific tweezers tip, such as a very narrow slanted tweezers tip that measures about 0.0625″ in width while another side can have a slanted tweezers tip that may measure about 0.200″ in width. Additionally, other embodiments may encompass a light feature to illuminate areas of interest such as shadow areas around the eyes or ears, etc. In some embodiments, the light is adapted to pivot or spin for better illumination. In still another embodiment, a magnifying lens may be incorporated to pivot and spin into position over the other tips to allow one to magnify the area of interest.

The tool may have varying surface treatments to provide improved grip for tweezing functions. Exemplary surface treatments may include finger concavities for finger placement, stamped surface textures to improve grip, or soft surface textures to enhance the controlled grip. Santoprene or TPE resins may be over-molded onto the tweezer plates or attached through the use of adhesives. The soft texture can be attached through the use of numerous mechanical attachments such as molded plugs on the soft texture component that snaps into the tweezer plates.

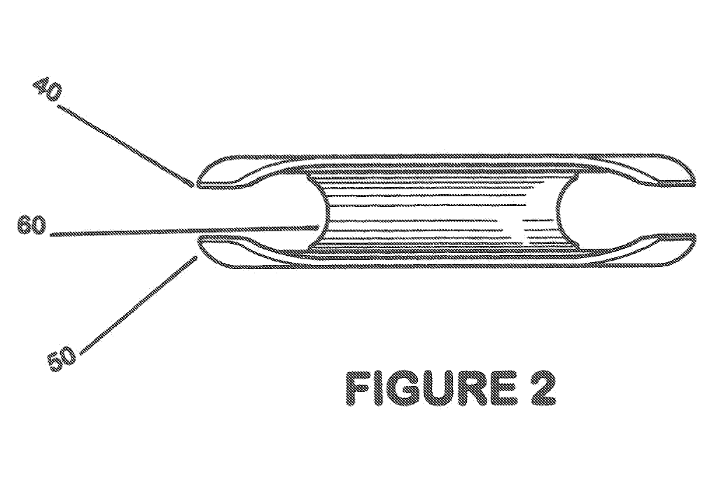

The present invention is also designed to incorporate a spring steel component formed for the flexible movement which allows the pivoting articulation required in applying pinching force upon an area of interest. The spring steel component may have a TPE “gasket” or soft material “sandwiched” in between the tweezers plates 40 and 50 as shown in FIG. 2 as gasket 60. Gasket 60 is designed to protect the spring from debris.

The multi-tweezer tool is preferably comprised of two stamped and formed metal tweezer plates 40 and 50 that are mirror images of each other to align the specific tip with the corresponding tip of the other tweezer plate when assembled. The flexible spring component is comprised of two separately stamped and formed spring steel pieces measuring approximately 0.020″ to 0.050″ in material stock thickness. The thickness of the stock of spring may also depend on the length of the tweezers “legs” to the tip and the thickness of the tweezer components, so the spring steel measurement may be revised per the design. In a preferred embodiment, the spring depression force for the tweezers may be between about 1 to about 2.5 lbs. of force as measured from the point where the fingers are positioned to depress the tweezers as measured from the tips inward. This dimension may range from about 0.500″ to about 0.750″ for optimal comfort and function. In this embodiment, the force was measured utilizing a 0.3 mm thick stamped & formed metal stock spring and the distance of depression at the tweezers tips to range from about 0.250″ to about 0.375″.

The spring components may be stamped from a single piece of spring steel that is engineered with three specific characteristics. The first characteristic of the spring is a base portion that can be adapted to be spot-welded later to one of the tweezer plate halves. The second characteristic is preferably the actual spring or flexible portion of the component. The third characteristic is the top spot-welding flat plate portion that will be used to bind the two spring components together in the assembly process. Both of the spring steel pieces may be positioned in the middle of a tweezer plate half utilizing a specifically designed alignment fixture that holds the components for the spot-welding process and than those two components will be spot-welded together forming a sub-assembly. A thermoplastic elastomer or TPE gasket 60 may then be positioned around the spring of a tweezer subassembly, placed in another specifically designed holding fixture and than the other mirror tweezer subassembly may be positioned and aligned with the pre-assembled tweezer within the holding fixture. Correct alignment and orientation are very important. The two subassemblies preferably trap the TPE “gasket” component when the two subassemblies are spot welded together. The spot weld is made possible by a stamped through-hole on both tweezer plates allowing access to the spring steel components and their top spot-welding plates. The spot-welding operation securely binds the two subassemblies together. The final assemble step may be the application of a logo plate on either side of the tweezer and may cover the through-hole that allowed access for the spot-welding operation.

While the present invention has been described with reference to specific examples, which are intended to be illustrative only and not to be limiting of the invention, it will be apparent to those of ordinary skill in the art that changes, additions or deletions may be made to the disclosed embodiments without departing from the spirit and scope of the invention.

We claim:

1. A multi-tool with more than one side, wherein said multi-tool is designed to perform one or more of the following functions
Dig;
Pull;
Squeeze;
Magnify; and
Illuminate.

2. The multi-tool of claim 1, wherein said multi-tool comprises two plates joined by a spring.

3. The multi-tool of claim 2, wherein the spring force between said two plates is about 1.0 to about 2.5 lbs.

4. The multi-tool of claim 1 wherein said dig functionality is performed by a tweezer tip with a sharp end.

5. The multi-tool of claim 1, wherein said pull functionality is performed by a flat perpendicular pulling tweezer.

6. The multi-tool of claim 1, wherein said squeeze functionality is performed by a rounded end tweezer.

7. The multi-tool of claim 1, wherein said illuminate function is performed by a light.

8. The multi-tool of claim 7, wherein said light is adapted to pivot.

9. The multi-tool of claim 2, wherein said plates have a surface coating for better grip.

10. A multi-faceted tool comprising
a first side adapted to dig into a users skin having a sharp-tipped tweezer
a second side adapted to pull from a user's skin having a flat perpendicular tweezer; and
a third side adapted to squeeze a user's skin having a rounded end tweezer.
